(19) United States
(12) Patent Application Publication
(10) Pub. No.: US 2021/0125396 A1
(43) Pub. Date: Apr. 29, 2021

(54) SYSTEMS AND METHODS FOR GENERATING THREE-DIMENSIONAL MEDICAL IMAGES USING RAY TRACING

(71) Applicant: Hyperfine Research, Inc., Guilford, CT (US)

(72) Inventors: Scott Martin, Salem, CT (US); Prantik Kundu, Guilford, CT (US)

(21) Appl. No.: 17/078,432

(22) Filed: Oct. 23, 2020

Related U.S. Application Data
(60) Provisional application No. 62/926,409, filed on Oct. 25, 2019.

(51) Int. Cl. G06T 15/06; G16H 30/20; G16H 30/40; G06T 7/00

(52) U.S. Cl. CPC G06T 15/06; G16H 30/20; G06T 2207/10088; G06T 7/0012; G16H 30/40

(57) ABSTRACT

Techniques for generation of three-dimensional (3D) medical images. The techniques include: receiving, via at least one communication network, image data obtained by at least one medical imaging device; generating, using ray tracing, the 3D medical image based on the image data obtained by the at least one medical imaging device; and outputting the 3D medical image.

### CROSS-REFERENCE TO RELATED APPLICATIONS

This application claims the benefit under 35 U.S.C. § 119(e) of U.S. provisional patent application Ser. No. 62/926,409, entitled “SYSTEMS AND METHODS FOR RENDERING MAGNETIC RESONANCE IMAGES USING RAY TRACING”, filed Oct. 25, 2019 under Attorney Docket No. 00354.70048US00, which is incorporated by reference in its entirety herein.

### FIELD

The present disclosure relates generally to image generation techniques based on data obtained using one or more medical imaging devices and, more specifically, to systems and methods for generating a three-dimensional (3D) medical image using ray tracing.

### BACKGROUND

Medical image data may be obtained by performing diagnostic medical imaging, such as magnetic resonance imaging, on subjects (e.g., patients) to produce images of a patient's anatomy. Medical image data can be obtained by a number of medical imaging devices, including magnetic resonance imaging (MRI) devices, computed tomography (CT) devices, optical coherence tomography (OCT) devices, positron emission tomography (PET) devices, and ultrasound imaging devices, for example.

### SUMMARY

Some embodiments provide for a system for generating a three-dimensional (3D) medical image, the system comprising: a mobile computing device comprising at least one computer hardware processor; and at least one non-transitory computer readable storage medium having encoded thereon executable instructions that, when executed by the at least one computer hardware processor, cause the mobile computing device to perform: receiving, via at least one communication network, image data obtained by at least one medical imaging device; generating, using ray tracing, the 3D medical image based on the image data obtained by the at least one medical imaging device; and outputting the 3D medical image.

Some embodiments provide for a method for generating a three-dimensional (3D) medical image comprising: receiving, with at least one computer hardware processor of a mobile computing device via at least one communication network, image data obtained by at least one medical imaging device; generating, using the at least one computer hardware processor to perform ray tracing, the 3D medical image based on the image data obtained by the at least one medical imaging device; and

Some embodiments provide for at least one non-transitory computer readable storage medium having encoded thereon executable instructions that, when executed by at least one computer hardware processor of a mobile computing device, cause the mobile computing device to perform: receiving, via at least one communication network, image data obtained by at least one medical imaging device; generating, using ray tracing, a three-dimensional (3D) medical image based on the image data obtained by the at least one medical imaging device; and outputting the 3D medical image.

### BRIEF DESCRIPTION OF THE DRAWINGS

Various aspects and embodiments of the disclosed technology will be described with reference to the following figures. It should be appreciated that the figures are not necessarily drawn to scale. For purposes of clarity, not every component may be labeled in every drawing.

FIG. 1 illustrates an example system for generating a three-dimensional medical image, in accordance with some embodiments of the technology described herein.

FIG. 2 illustrates an example process for generating a three-dimensional medical image, in accordance with some embodiments of the technology described herein.

FIGS. 3A-3B illustrate an example process for generating a three-dimensional medical image using ray tracing, in accordance with some embodiments described herein.

FIG. 4A illustrates an example process for generating shadows in a three-dimensional medical image, in accordance with some embodiments of the technology described herein.

FIG. 4B illustrates an example process for determining an amount a light source is occluded from a voxel, in accordance with some embodiments of the technology described herein.

FIG. 5A illustrates an example medical image obtained using ray tracing, in accordance with some embodiments of the technology described herein.

FIG. 5B illustrates an example graphical user interface for viewing and interacting with the example medical image of FIG. 5A, in accordance with some embodiments of the technology described herein.

FIGS. 5C-5E illustrates an example of graphical user interface illustrating medical images in different views, in accordance with some aspects of the technology described herein.

FIG. 6 illustrates example components of a magnetic resonance imaging device, in accordance with some embodiments of the technology described herein.

FIG. 7 illustrates a block diagram of an example computer system, in accordance with some embodiments of the technology described herein.

### DETAILED DESCRIPTION

Aspects of the present application relate to systems and methods for generating three-dimensional (3D) medical images using ray tracing. For example, in some embodiments, the systems and methods described herein may be used to generate 3D medical images of a patient anatomy based on magnetic resonance imaging (MRI) data or any other suitable type of medical imaging data (e.g., computed tomography (CT) imaging data, optical coherence tomography (OCT) imaging data, positron emission tomography (PET) imaging data, ultrasound imaging data). The generated image may be photo-realistic to give a viewer the perception that the patient anatomy rendered in the image is a real object in true space.

Conventional techniques for rendering high-quality 3D images typically use using custom-built graphics software, which performs extensive physics-based computations executing which is computationally expensive, requiring substantial processor and/or memory resources. Often, specialized hardware is utilized such as, for example, multiple graphics processing units (GPUs). As a result, in practice, such conventional techniques are implemented using high-performance computer workstations and/or cloud computing infrastructure. Such computing resources are expensive and lack portability, thus limiting the accessibility of 3D image generation techniques where such techniques would otherwise be useful to assist a medical professional in diagnosing and/or treating a patient.

Ray tracing is one example of a technique for generating a photo-realistic 3D image. Ray tracing is a class of techniques that generate images in part by simulating the natural flow of light in a scene from one or multiple light sources in the scene. Ray tracing techniques may involve production of multiple rays for each pixel being rendered. The rays may be traced through multiple “bounces”, or intersections with one or more objects in the scene, in part, based on laws of optics concerning reflection, refraction, and surface roughness. A surface illumination may be determined at a point at which the ray encounters a light source or a point after a predefined number of bounces. As such, ray tracing techniques may provide highly realistic 3D images, but generally carry a high computational cost which requires the use of high-performance computer workstations and/or cloud computing infrastructure.

Mobile computing devices (e.g., smartphones, tablets, laptops, personal digital assistants, etc.) lack the sufficient processor and memory resources required to perform conventional 3D image generation techniques, such as conventional techniques for generating a 3D image using ray tracing. As such, high quality rendering of 3D images is generally not available on mobile computing devices. Even in instances where 3D image generation would be possible on a mobile computing device, for example using techniques other than ray tracing, the lengthy processing time would render doing so impractical. Instead, with conventional techniques, mobile computing devices are only capable of streaming a pre-generated image (e.g., a 3D image generated by a high-performance computer workstation and/or cloud computing infrastructure transmitted to a mobile computing device) and do not themselves perform image generation.

The inventors have recognized that access to high-quality 3D images of patient anatomy at the point-of-care would be beneficial to medical professionals in order to most effectively treat and/or diagnose a patient. For example, a medical professional may benefit from viewing and interacting with a 3D image of the patient anatomy on a mobile computing device. In particular, a medical professional may benefit from interacting with a 3D medical image (e.g., by rotating, translating, zooming in or out on, and/or cross-sectioning an object illustrated by the 3D medical image) via a graphical user interface.

In order to preserve the photo-realistic depiction of the patient anatomy, an updated 3D medical image may be generated in response to interaction by the user. However, as described above, mobile computing devices are not able to generate such high-quality imagery using conventional image rendering techniques. Rather, mobile computing device must communicate with a high-performance computer workstation to relay an instruction for the high-performance workstation to generate an updated 3D medical image reflecting the user interaction. The updated image must then be transmitted back to the mobile computing device for viewing and/or further interaction by the medical professional. This process must occur every time the medical professional interacts with the 3D medical image, creating significant delays.

Thus, the inventors have developed a technique for generating 3D medical images that can be performed on a user's mobile computing device in real-time (e.g., in response to receiving at least a portion of image data, in response to receiving an instruction to generate the 3D medical image, etc.). Thus, as opposed to conventional techniques which only enable a mobile computing device to stream a pre-generated image rendered by a high-performance workstation and transmitted to the mobile computing device, a mobile computing device may itself generate 3D medical images using the techniques described herein eliminating the necessity for the mobile computing device to rely on high-performance workstations to perform image generation. Accordingly, a medical professional may view and interact with a 3D medical image, and the mobile computing device may generate an updated 3D medical image in real time (e.g., in response to receiving the interaction) reducing the delays associated with prior techniques.

In some embodiments, the image generation techniques described herein may be performed by executing software with a mobile computing device (or any other suitable device). In some embodiments, the software may be sent to a second device via any suitable communication network (e.g., internet, cellular, wired, wireless, etc.).

The software may be any suitable type of software. For example, in some embodiments, the software may be executable by a web browser executing on a mobile computing device. The software may be written in any suitable programming language. For example, in some embodiments, at least a port of the software may be writing in JAVA, JAVASCRIPT, COFFEESCRIPT, PYTHON, or any other suitable programming language that can be executed by a web browser or compiled to a language that can be executed by the web browser.

The techniques developed by the inventors and described herein may be used to generate high-quality 3D medical images on mobile computing devices using ray tracing. These techniques provide an improvement to medical imaging technology because they enable use of ray tracing to generate photo-realistic medical images on mobile computing devices, which were previously not able to do so. Consequently, the techniques described herein enable medical professionals to view, interact with, and/or share photo-realistic images on a mobile computing device at the point-of-care, which facilitates treatment and/or diagnosis of patients.

It should be appreciated that the 3D image generating techniques developed by the inventors also constitute an improvement to computer technology. Conventionally, as described above, high-quality rendering of medical images would be performed on remote computing resources (e.g., high performance workstation and/or cloud-computing infrastructure), which requires transmission of a large amount of data on networks (e.g., requests to generate and/or update medical images, the generated images, and/or updates to generated images). Rendering high-quality images, locally, on a mobile computing devices saves network resources and reduces the load of remote computing resources as well, both of which constitute improvements to computer technology.

Thus, aspects of the present disclosure relate to systems and methods for generating one or more 3D medical images using ray tracing. According to some aspects of the technology described herein, there is provided a system for generating a 3D medical image, the system comprising a mobile computing device (such as a mobile phone, for example) comprising at least one computer hardware processor, and at least one non-transitory computer readable storage medium having encoded thereon executable instructions that, when executed by the at least one computer hardware processor, cause the mobile computing device to perform (1) receiving, via at least one communication network, image data (e.g., MRI data, CT imaging data, OCT imaging data, PET imaging data, ultrasound imaging data) obtained by at least one medical imaging device (e.g., an MRI device, a CT device, an OCT device, a PET device, an ultrasound device); (2) generating, using ray tracing, the 3D medical image based on the image data obtained by the at least one medical imaging device; and (3) outputting the 3D medical image (e.g., displaying the 3D medical image on the mobile computing device, transmitting the 3D medical image to a second device external to the mobile computing device for viewing by a user of the second device, saving the 3D medical image to a memory of the mobile computing device, etc.). In some embodiments, the generating is performed in response to receiving at least a portion of the image data.

In some embodiments, the executable instructions comprise JAVASCRIPT instructions for generating the 3D medical image using ray tracing. In some embodiments, the executable instructions comprise GL SHADING LANGUAGE. In some embodiments, the generating is performed by executing the JAVASCRIPT and/or GL SHADING LANGUAGE instructions on a web browser executing on the mobile computing device.

In some embodiments, the method further comprises generating a two-dimensional image based on the 3D medical image.

In some embodiments, the ray tracing comprises: (1) generating at least one ray; (2) determining values for at least one characteristic (e.g., gradient, luminosity) at locations along the ray; and (3) generating a pixel value based on the determined values for the at least one characteristic at the locations along the ray. The ray tracing may further comprise (4) determining an amount by which light source is occluded from at least one location along the ray.

In some embodiments, the executable instructions further cause the mobile computing device to generate a graphical user interface (GUI) for viewing the 3D medical image. In some embodiments, the GUI is configured to allow a user to provide an input indicative of a change to be made (e.g., rotating, translating and/or cross-sectioning an object depicted by the 3D medical image) to the 3D medical image. In some embodiments, the executable instructions further cause the mobile computing device to generate, using ray tracing, an updated 3D medical image in response to receiving the input provided by the user.

In some embodiments, the image data comprises MRI data, CT imaging data, OCT imaging data, PET imaging data, and/or ultrasound imaging data.

In some embodiments, the mobile computing device is battery powered. In some embodiments, a display and the at least one computer hardware processor of the mobile computing device is disposed in a same housing.

According to some aspects of the technology described herein, there is provided a method for generating a 3D medical image comprising (1) receiving, with at least one computer hardware processor of a mobile computing device (such as a mobile phone, for example) via at least one communication network, image data obtained by at least one medical imaging device (e.g., a magnetic resonance imaging device); (2) generating, using the at least one computer hardware processor to perform ray tracing, the 3D medical image based on the image data obtained by the at least one medical imaging device; and (3) outputting the 3D medical image.

In some embodiments, there is provided at least one non-transitory computer readable storage medium having encoded thereon executable instructions that, when executed by at least one computer hardware processor of a mobile computing device, cause the mobile computing device to perform (1) receiving, via at least one communication network, image data obtained by at least one medical imaging device; (2) generating, using ray tracing, a 3D medical image based on the image data obtained by the at least one medical imaging device; and (3) outputting the 3D medical image.

The aspects and embodiments described above, as well as additional aspects and embodiments, are described further below. These aspects and/or embodiments may be used individually, all together, or in any combination, as the technology is not limited in this respect.

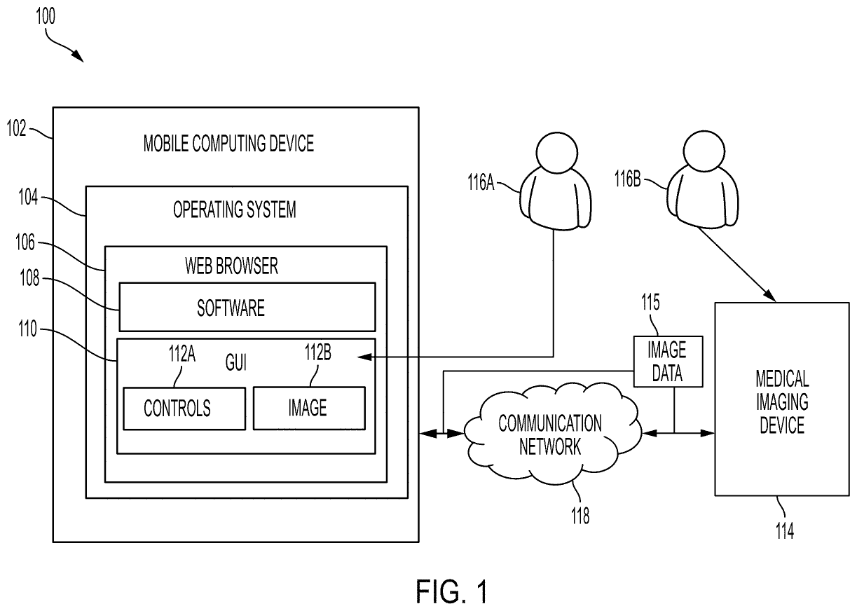

FIG. 1 illustrates an example system 100 for generating a 3D medical image, in accordance with some embodiments of the technology described herein. As shown in FIG. 1, system 100 includes a mobile computing device 102 such as a mobile phone, laptop, tablet, personal digital assistant, etc. The mobile computing device may be configured to generate one or more 3D medical images using ray tracing from data obtained by a medical imaging device 114.

In some embodiments, the mobile computing device may be integrated with a display. In some such embodiments, the display, and any processing units of the mobile computing device may be disposed in the same housing. In some embodiments, the display may be a touch screen.

In some embodiments, the mobile computing device may have one or more central processing units. In some embodiments, the mobile computing device may not include a graphics processing unit. Though, in other embodiments, the mobile computing device may include a graphics processing unit, as aspects of the technology described herein are not limited in this respect.

In some embodiments, the mobile computing device may be battery-powered and include one or more batteries. In some embodiments, the mobile computing device may operate entirely on power drawn solely from the one or more batteries without being connected to wall power.

In some embodiments, the system further comprises a medical imaging device 114. The mobile computing device 102 may receive image data 115 for generating the 3D medical image using ray tracing obtained by the medical imaging device 114. The medical imaging device 114 may be a magnetic resonance imaging device, a computed tomography device, an optical coherence tomography device, a positron emission tomography device, an ultrasound device, and/or any other suitable medical imaging device. The image data 115 may be representative of a patient anatomy for which generation of a 3D image is desired. Although in the illustrated embodiment a single medical imaging device 114 is shown, there may be provided multiple medical imaging devices for obtaining image data 115. A user 116B may interact with the medical imaging device 114 to control one or more aspects of imaging performed by the medical imaging device.

In some embodiments, the mobile computing device 102 receives the image data 115 from the medical imaging device(s) 114 directly. In some embodiments, the mobile computing device 102 receives the image data 115 from one or more other devices. In some embodiments, image data 115 obtained by the medical imaging device(s) is stored in a memory, and is retrieved by the processor 104. In some embodiments, the image data 115 may be received by the mobile computing device 102 via at least one communication network 118. In other embodiments, for example, the image data 115 may be received by the mobile computing device 102 from a memory of the mobile computing device.

The communication network 118 may be any suitable network through which the mobile computing device 102 can receive medical imaging data including, for example, medical imaging data collected by the medical imaging device(s) 114. In some embodiments, the communication network 118 is the Internet. In some embodiments, the communication network may be a local area network (LAN) a wide area network (WAN) or any suitable combination thereof. For example, the communication network 118 may be an internal communication network of a hospital. In some embodiments, the communication network 118 may have one or more wired links, one or more wireless links, and/or any suitable combination thereof.

The image data 115 may be obtained by a medical imaging device(s). For example, in some embodiments, the image data 115 comprises MRI data obtained by an MRI device, CT data obtained by a CT imaging device, OCT data obtained by an OCT imaging device, PET data obtained by a PET imaging device, ultrasound data obtained by an ultrasound imaging device, and/or any other type of image data 115. Image data 115 may be in any suitable format (e.g., Analyze format, Minc format, Neuroimaging Informatics Technology Initiative (NIfTI) format, Digital Imaging and Communications in Medicine (DICOM) format, Nearly Raw Raster Data (NRRD) format, or any other suitable format). The data may be compressed using any suitable compression scheme, as aspects of the technology described herein are not limited in this respect.

As described herein, the mobile computing device 102 may generate a 3D medical image based on the image data 115 according to the ray tracing techniques described herein, by contrast to previous techniques which only allowed for streaming a pre-generated 3D medical image. In some embodiments, the mobile computing device 102 may generate the 3D medical image in response to receiving the image data 115. In some embodiments, the mobile computing device 102 may receive a portion but not all of the image data 115 and begin generating the 3D medical image based on the portion of image data 115 received.

For example, the image data 115 obtained by the medical imaging device(s) 114 may include a first set of data and a second set of data. In such embodiments, the mobile computing device 102 may receive the first set of data and generate a 3D medical image based on the first set of data prior to completing receipt of the second set of data. In some embodiments, the mobile computing device 102 may receive the second set of data and update the 3D medical image generated based on the first set of data using the second set of data. In this way, a user need not wait for all of the image data to be received in order to start viewing a visualization of the image data. This may be helpful especially in circumstances where the image data is large and may take a long time to download. Such a process may be referred to herein as streaming rendering.

For example, a process for streaming rendering may be performed using MR image data. As one example, in such embodiments, the first set of image data may comprise a first set of MR slices, and the second set of data may comprise a second set of MR slices. In another embodiment, the first set of data may comprise MR data collected by sampling a first part of k-space (i.e., the spatial frequency domain), and the second set of data may comprise MR data collected by sampling a second part of k-space. For example, the first part of k-space may include a central region of k-space including low spatial frequency data, for which rendering would give a smoothed out visualization of the patient's anatomy. The second part of k-space may include a region complementary to the central region of k-space. Updating a visualization obtained based on the first set of data comprising the MR data collected by sampling the first part of k-space by using the second set of data may introduce high-frequency features and sharpen the details of the visualization of the patient's anatomy. It should be appreciated that the image data may comprise any suitable number of parts, and aspects of the technology are not limited in this respect to the first and second data sets described herein.

In some embodiments, the mobile computing device 102 may be communicatively coupled (e.g., via communication network 118) to an operating system 104. The operating system may comprise any suitable operating system, for example, ANDROID, IOS, MACOS, MICROSOFT WINDOWS, or any other operating system. The operating system 104 may execute a web browser 106, as shown in FIG. 1. Any suitable web browser may be used (e.g., GOOGLE CHROME, INTERNET EXPLORER, MICROSOFT EDGE, SAFARI, FIREFOX, etc.).

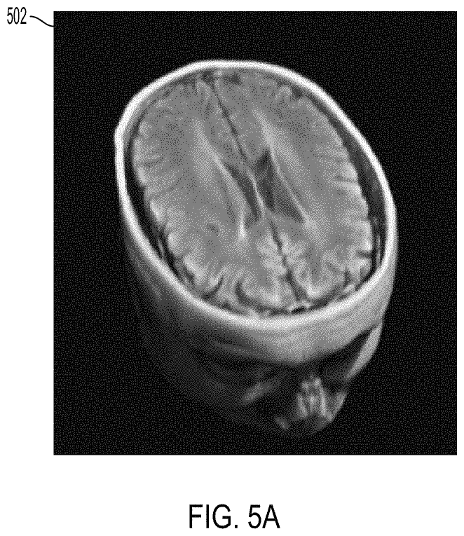

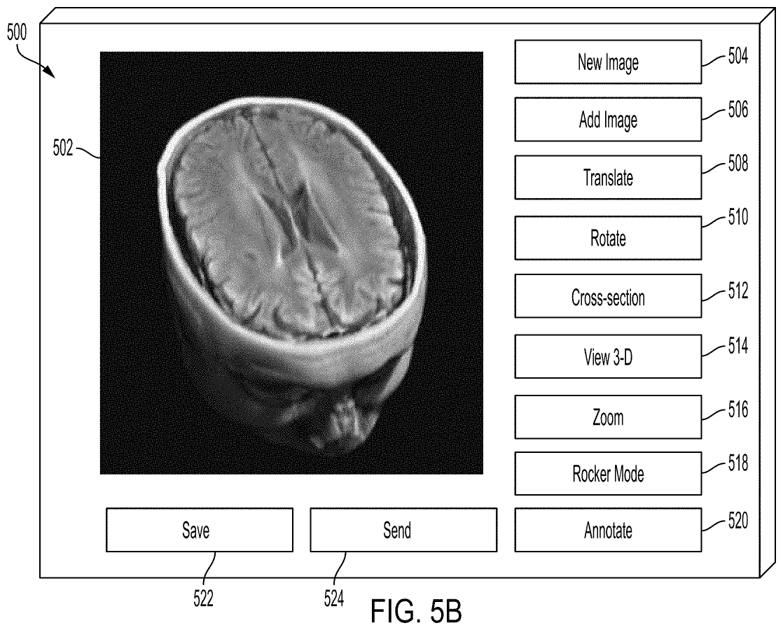

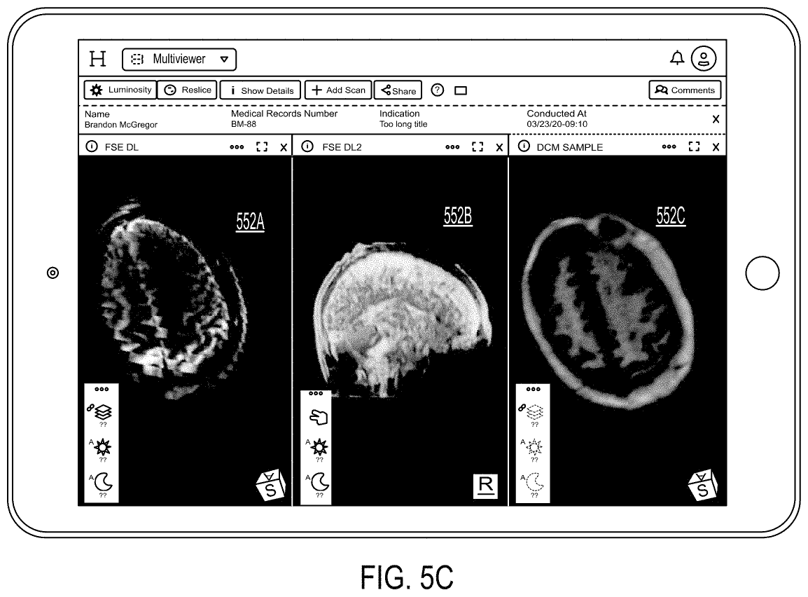

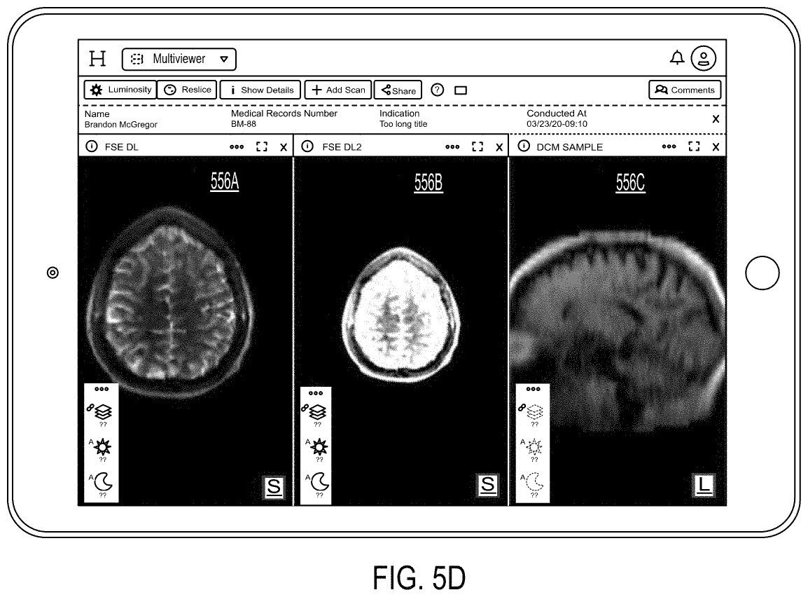

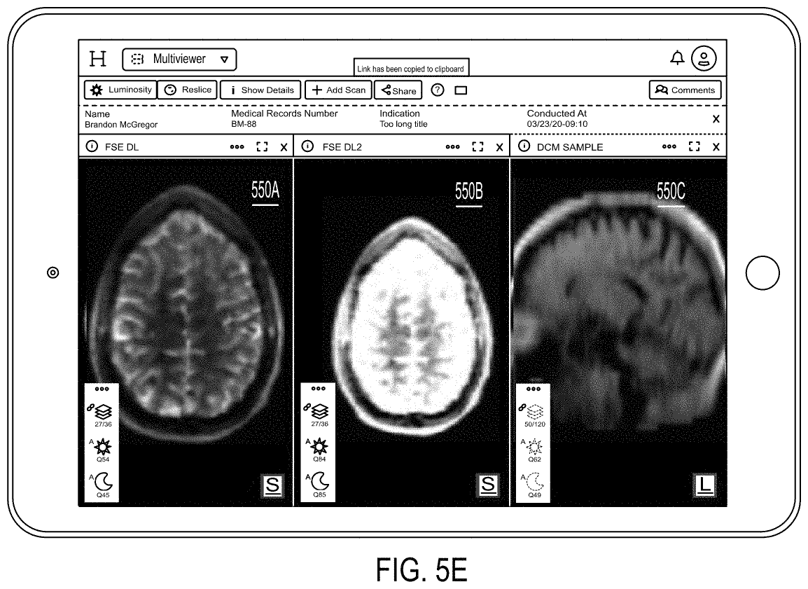

The web browser 106 may generate a graphical user interface (GUI) 110. As described herein, the GUI 110 may display a medical image 112B (e.g., a 3D medical image generated by the mobile computing device 102). The GUI 110 may include controls which allow a user 116A to interact with the image 112B and/or to control one or more aspects of image generation. For example, as described herein, controls 112A of GUI 110 may enable a user 116A to translate, rotate, zoom in or out on, and/or cross-section an object illustrated by the medical image 112B. Examples of GUI controls are described herein, including with reference to FIGS. 5A-5E.

Software 108 may be executed on the web browser 106. For example, the software 108 may comprise executable instructions, that, when executed by the mobile computing device 102, cause the mobile computing device 102 to generate a 3D medical image. In some embodiments, the software 108 comprises executable instructions for generating the GUI 110. In some embodiments, the software 108 comprises executable instructions written in JAVASCRIPT, JAVA, COFFEESCRIPT, PYTHON, or any other suitable programming language. In some embodiments, the software 108 comprises executable instructions that are compiled into another programming language and/or instruction format second format executable by the web browser 106. For example, the software 108 may comprise executable instructions written in COFFEESCRIPT, PYTHON, RUBY, PERL, JAVA, C++, C, BASIC, GL SHADING LANGUAGE (GLSL), or any other suitable format, that compiles to another programming language and/or instruction format (e.g., JAVASCRIPT) executable by the web browser 106.

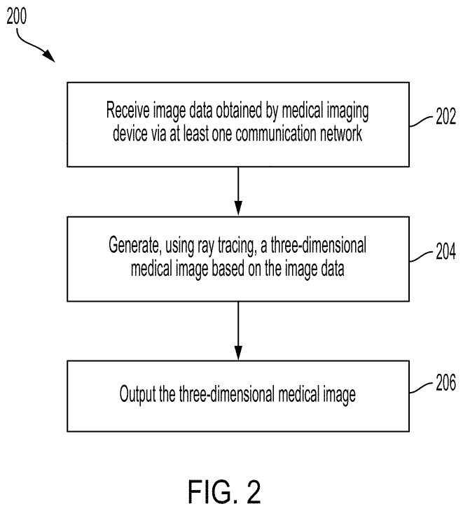

FIG. 2 illustrates an example process for generating a 3D medical image, in accordance with some embodiments of the technology described herein. The example process 200 may be performed by the mobile computing device 102 of FIG. 1.

Process 200 begins at act 202 where image data obtained by a medical imaging device is received, for example, via at least one communication network. As described herein, the medical imaging device may comprise one or more devices for obtaining image data representative of a patient anatomy for which generation of a 3D medical image is desired. Examples of medical imaging devices are provided herein.

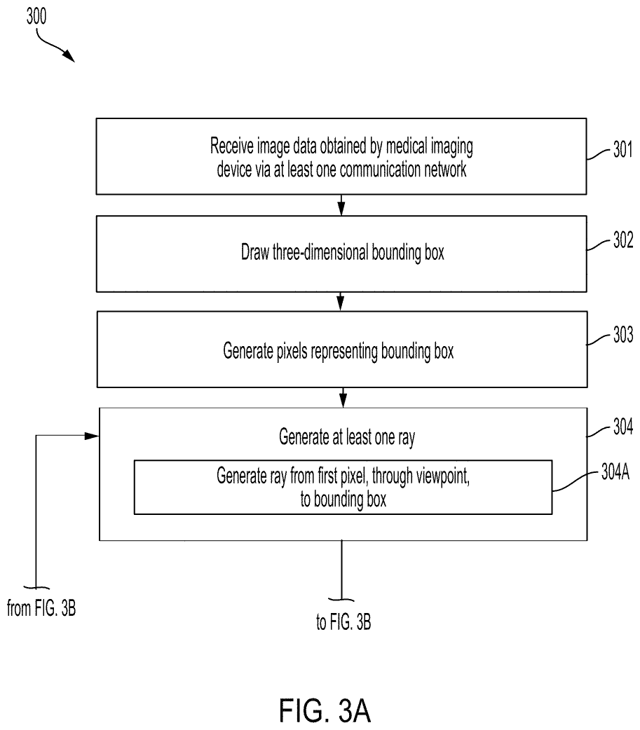

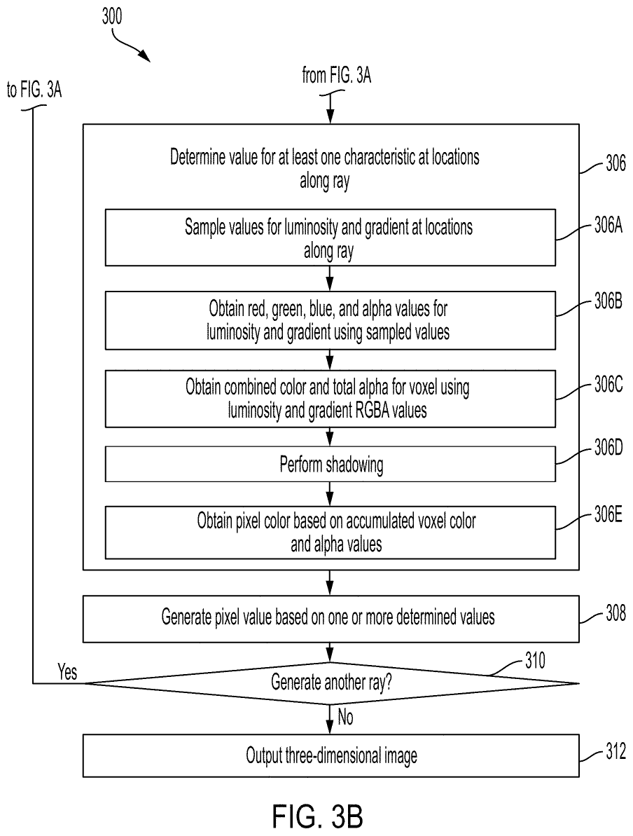

At act 204, a 3D medical image may be generated, using ray tracing, based on the image data received at act 202. For example, act 204 may be performed in accordance with process 300 described herein with reference to FIG. 3.

At act 206, the generated 3D medical image may be output by the processor. In some embodiments, outputting the medical image may comprise displaying the 3D medical image via a display (e.g., a display integrated with a mobile computing device, such as a mobile phone, tablet, etc.).

In some embodiments, outputting the medical image comprises saving the medical image to a memory. For example, outputting the medical image may comprise saving the medical image to a memory of the mobile computing device performing process 300. As another example, outputting the medical image may comprise saving the medical image to an external storage (e.g., cloud-based storage and/or any other suitable external storage) external to the mobile computing device performing process 300. As yet another example, outputting the medical image may comprise saving the medical image to both a memory of the computing device performing process 300 and to external storage external to the mobile computing device.

In some embodiments, outputting the medical image comprises transmitting the medical image to one or more second devices. For example, the one or more second devices may be a mobile computing device, a desktop computer, high-performance workstation, or other suitable device. In some embodiments, the one or more second devices may be operated by a medical professional with whom a user of the mobile computing device desires to share the medical image (for example, to obtain the input of the medical professional). In some embodiments, the one or more second devices may be operated by an individual performing imaging (for example, to determine whether additional imaging should be performed).

FIGS. 3A-3B illustrate an example process 300 for generating a 3D medical image using ray tracing, in accordance with some embodiments described herein. In particular, process 300 provides an example embodiment for generating, using ray tracing, the three-dimensional medical image based on the image data at act 206 of process 200. Process 300 may be performed by the mobile computing device 102 described with reference to FIG. 1 or any other suitable mobile computing device.

Process 300 begins at act 301, where image data obtained by a medical imaging device(s) may be received. As described herein, the medical imaging device may comprise one or more devices for obtaining image data representative of a patient anatomy for which generation of a 3D medical image is desired.

At act 302, a 3D bounding box is drawn. The 3D bounding box may establish a region that is to contain the object being represented by the 3D medical image. In some embodiments, the 3D bounding box comprises a number of triangles (e.g., twelve triangles which may, in some embodiments, be arranged to form a cube or a rectangular box, for example). In some embodiments, the 3D bounding box is drawn via a JAVASCRIPT WebGL interface.

At act 303, a plurality of pixels may be generated representing at least a portion of the bounding box. In particular, at act 303, a graphics card of the mobile computing device may generate the plurality of pixels by performing rasterization. For example, in some embodiments, only a portion of the triangles generated at act 302 are converted into pixels (e.g., only the triangles facing a viewpoint).

At act 304, at least one ray (e.g., an initial ray) may be generated. In some embodiments, act 304 is performed according to act 304A. At act 304A, an initial ray may be generated for a first pixel. It should be appreciated that multiple initial rays for respective ones of the plurality of pixels may be generated in parallel, although act 304A is described with reference to a first pixel. For example, act 304A may be performed for each pixel generated at act 303. The initial ray may be generated for the first pixel from the first pixel's location in world space, through a viewpoint (e.g., a camera), and to the bounding box.

The process 300 may then move to act 306, where a value for at least one characteristic at locations along the initial ray may be determined. In some embodiments, the at least one characteristic may be used to determine a color value to assign to the first pixel. The at least one characteristic may be any suitable number and/or type of characteristic (for example, a red, green, and/or blue value, a transparency value, etc.). Acts 306A-306E provide one example of a process for determining a value for at least one characteristic at locations along the initial ray.

At 306A, values for luminosity and gradient may be sampled at locations along the initial ray. In particular, luminosity and gradient values may be obtained, based on medical image data (e.g., MR image data, in some embodiments) received by the mobile computing device, spaced along multiple locations of the initial ray generated at act 304. The multiple locations may be spaced evenly along the initial ray, in some embodiments. In other embodiments, the multiple locations may be spaced non-uniformly, as aspects of the technology are not limited in this respect. Any suitable spacing may be used, dependent on desired resolution and computational cost. In some embodiments, the spacing may be adjusted based on the frame rate of the web browser executing the software (e.g., by increasing spacing for lower frame rates and increasing spacing for higher frame rates). The respective locations along the initial ray may be referred to as voxels.

A luminosity value may be a scalar value representative of an intensity at a point in the original object volume and is rendered as a block of color with an associated translucency. Gradient values may include a vector with three values representative of a change in luminosity in three dimensions and is rendered as a surface at locations where there are sharp transitions (e.g., transitions between values that change by more than a threshold amount between pixels) between luminosity values which may assist in illustrating an underlying structure of the object being rendered.

In some embodiments, gradient values may be determined from luminosity values in the object volume and stored in an optimized format as part of a texture. As described herein, gradient values may include a three-dimensional vector. In some embodiments, the three gradient vector values may be normalized, biased, and/or stored as part of red, green, and blue channels of the texture. In some embodiments, an alpha value may be stored containing the normalized magnitude of the gradient values. The inventors have recognized that storing gradient values in this format may significantly decrease the time required to render the 3D medical image (e.g., by a factor of six).

At act 306B, the sampled values for luminosity and gradient may be used to obtain red, green, blue, and alpha values for each of luminosity and gradient. The red, green, blue, and alpha values may be obtained using one or more look-up tables. In some embodiments, there is a separate look-up table for each of luminosity and gradient. The inventors have recognized that storing luminosity and gradient values in separate look-up tables reduces the memory required to perform act 306B, as well as decreasing the difficulty of modifying each look-up table when desired. The look-up tables may be in any suitable format. In some embodiments, the look-up tables are stored in a memory (e.g., a memory of the computing device, a remote memory accessible by the mobile computing device). In some embodiments, the look-up tables may be customizable by a user. For example, a user may select a particular set of look-up tables depending on the object being rendered (e.g., a type of tissue, a portion of the patient anatomy).

At act 306C, values for totalAlpha and combinedColor may be obtained using the alpha values for luminosity and gradient determined at act 306B. First, the luminosity and gradient alpha values may be merged into a value for totalAlpha using the following formula:

totalAlpha=lumColor.a+gradColor.a−(lumColor.a*gradCplor.a)

where lumColor.a and gradColor.a are the alpha values for luminosity and gradient, respectively, obtained via the one or more look-up tables at act 306B. totalAlpha is representative of the transparency of the voxel. For example, a totalAlpha value closer to zero indicates that the voxel is relatively more transparent, while a totalAlpha value closer to 1 indicates that the voxel is relatively more opaque. A totalAlpha value equal to zero may indicate that the voxel is completely transparent (e.g., allowing light to pass through the voxel completely), while a totalAlpha value equal to 1 may indicate that the voxel is completely opaque (e.g., blocking all light from passing through the voxel).

Then, the totalAlpha value may be used along with the red, green, and blue (RGB) values for luminosity and gradient to obtain a combined color using the following formula:

$$combinedColor \cdot r g b = \frac{lumColor \cdot rgb * lumColor \cdot a + gradColor \cdot rgb * gradColor \cdot a}{t o talAlpha}$$

where lumColor.rgb and gradColor.rgb are the red, green, and blue values for luminosity and gradient, respectively, obtained via the one or more look-up tables at act 306B, lumColor.a and gradColor.a are the alpha values for luminosity and gradient, respectively, obtained via the one or more look-up tables at act 306B, and totalAlpha is obtained using the formula previously described herein. The combinedColor.rgb is representative of a red, green, and blue color value for the voxel having transparency of the voxel factored in.

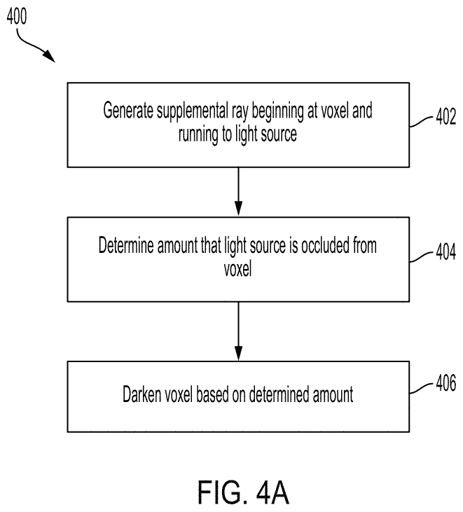

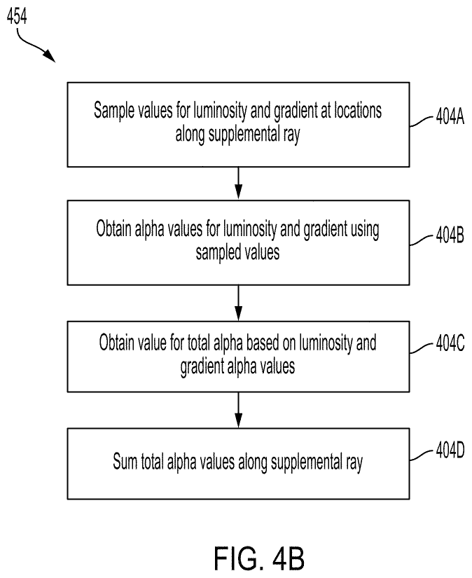

At act 306D, a process for generating shadows in the medical image may be performed. For example, shadowing may comprise determining an amount by which a light source is occluded from a pixel. Shadowing may be performed, in some embodiments, according to the example process 400 illustrated in FIG. 4A

At act 306E, a pixel color may be obtained based on accumulated color and alpha values for voxels along the initial ray. In particular, each voxel may have a combinedColor.rgb value obtained at act 306C. In some embodiments, the combinedColor.rgb value may be modified via the shadowing process at act 306D. The resulting color for each voxel may then referred to as the voxelColor having red, green, blue values referred to as voxelColor.rgb and an alpha value referred to as voxelColor.a which is equal to the totalAlpha obtained at act 306C.

All of the voxelColor.rgb values and the voxelColor.a values are summed up along the initial ray to obtain an accumulatedColor.rgb value for the pixel according to the following equation:

accumulatedColorᵢ.rgb=vec3(accumulatedColor_{i-1.rgb+(}1.0−accumulatedColorᵢ₋₁.a)*voxelColorᵢ.a*voxelColorᵢ.rgb)

where accumulatedColorᵢ₋ₗ.rgb is the composited total accumulatedColor.rgb as of voxel i, accumulatedColorᵢ₋₁.a is the composited total accumulatedColor.a as of voxel i, voxelColorᵢ.a is the voxelColor.a value at voxel i, and voxelColorᵢ.rgb is the voxelColor.rgb value at voxel i. The accumulatedColor.rgb,a values are accumulated for each voxel and fed into the equation for a subsequent voxel. AccumulatedColor.a is given by the following equation:

accumulatedColorᵢ.a=accumulatedColorᵢ₋₁.a*(1.0−voxelColorᵢ.a)+voxelColorᵢ.a

where accumulatedColorᵢ₋₁.a is the composited total accumulatedColor.a as of voxel i, and voxelColorᵢ.a is the voxelColor.a value at voxel i. The accumulatedColor.a value is accumulated for each voxel and fed into the equation for a subsequent voxel.

The accumulatedColor.rgb,a value may be representative of a color to be assigned to a respective pixel having translucency factored into the obtained value. At act 308, a pixel value (e.g., a color value such as the accumulatedColor.rgb,a value) may be generated for the first pixel.

In some embodiments, the acts 304-308 may be repeated as necessary for one or more of the remaining pixels generated at act 303. Together, the pixels may form a 3D image. Thus, at act 310, it may be determined whether to generate an additional ray beginning at the next pixel. If it is determined that an additional ray is to be generated, the process 300 returns through the yes branch to act 304. Otherwise, the process proceeds through the no branch to act 312. In some embodiments, generating another ray occurs in parallel with generating the initial ray. Thus, although the process 300 is illustrated as sequential, acts 304-308 may occur in parallel for each of the pixels generated at act 303.

At act 312, a 3D image (e.g., comprising the one or more pixels generated and colored at acts 304-308) may be output. For example, the outputting may comprise displaying the generated image via a 2-D or 3-D display. In some embodiments, outputting the image may comprise storing the generated image to a memory. In some embodiments, outputting the image may comprise transmitting the generated image to one or more second devices.

In some embodiments, the process 300 further comprises performing an additional optimization to the generated image by performing stippling. Stippling may better integrate each of the ray samples into the final image. In some embodiments, stippling is implemented in the form of a simple bias that is added to the location of the first sample value looked up by the for each ray (e.g., at act 306A), based on a Bayer stipple pattern. The effect of this stippling process may be to soften sharp transitions in the image at the potential sacrifice of in-plane resolution for depth resolution during rendering.

For example, as described herein, spacing of voxels along rays extending from a pixel to a viewpoint may depend on desired resolution and/or available frame rate. Lower frame rates may result in increased spacing of voxels to improve rendering speed. In some embodiments, stippling may be applied to reduce artifacts and/or improve apparatus image quality in instances where the spacing of voxels is increased.

As described herein, the process 300 for generating a 3D medical image using ray tracing may be performed by a mobile computing device. In some embodiments, the process 300 may be performed in response to receiving at least a portion of the image data. Further, in some embodiments, the process 300 may be repeated in response to receiving a user input indicating a change to be made to the 3D medical image (e.g., rotating the image, translating the image, cutting a plane of the image, etc.). The fast speed of the process 300 for generating the 3D medical image using ray tracing may be beneficial to medical professionals who may be viewing the generated medical images at the point-of-care, as described herein.

It should be appreciated that one or more of the acts of process 300 are optional and may be omitted. For example, in some embodiments, shadowing performed at act 306D is omitted. In some embodiments, only one of luminosity or gradient is sampled at locations along the initial ray at act 306A. In some embodiments, one or more other characteristics are additionally or alternatively sampled at act 306. Further, one or more additional or alternative calculations may be made at act 306, the technology is not limited to the calculations shown by way of example at acts 306A-306E.

Aspects of the techniques described herein allow for 3D image generation using ray tracing to be implemented on a mobile computing device without requiring significant delays in processing time. For example, at act 304, processing time may be reduced by only generating pixels representing the bounding box for portions of the bounding box which are visible to a user from a viewpoint. Thus, at most, half of the bounding box is converted into pixels resulting in a two-fold reduction in the number of pixels for which values are computed. At act 306D, processing time for performing shadowing may be reduced by ending summation of totalAlpha values of voxels along the supplemental ray once a threshold summed totalAlpha is reached (e.g., when there is already enough occlusion to block substantially all light from the light source such that the impact of additional voxel's opacity is negligible, as described herein). Furthermore, the calculations at act 306 to determine a value for at least one characteristic at locations along the ray may be made simpler by initially obtaining characteristic values from known information reflected in the image data (as opposed to generating such information without reference to the image data). Subsequently, pixel color may be determined by simple arithmetic using the obtained characteristic values. Thus, the computational cost of obtaining the pixel color values is low. Computational cost may be further reduced by controlling the density of sampling locations along the initial ray at act 306. As described herein, conventional techniques trace rays through multiple bounces with objects in the rendering space. In some embodiments, the techniques described herein do not calculate bounces of rays.

FIG. 4A illustrates an example process for generating shadows in a 3D medical image, in accordance with some embodiments of the technology described herein. As described herein, act 306 of process 300 may further include an additional process 400 for generating shadows in the 3D medical image.

As shown in FIG. 4, the process 400 begins at act 402, where a supplemental ray may be generated. For example, the supplemental ray may be generated running from the voxel for which shadowing is being performed, through a light source, to the bounding box. The supplemental ray may be generated running through the light source in order to determine whether light from the light source is partially or fully occluded from reaching the voxel of the initial ray. Doing so may indicate how much shadow is to be applied to the voxel of the initial ray.

Determining whether light from the light source is partially or fully occluded from reaching the voxel of the initial ray may depend on how transparent or opaque the voxels of the supplemental ray which are located between the light source and the voxel of the initial ray are. As such, at act 404, an amount that a light source is occluded from a voxel of the initial ray is determined. For example, the amount that the light source is occluded from the voxel of the initial ray may be determined by performing the process 454 illustrated in FIG. 4B.

Then, at act 406, the voxel of the initial ray may be darkened based on the amount determined at act 404. For example, at act 406, red, green, and blue values for the voxel of the initial ray may be darkened based on the amount determined at act 404. It should be appreciated that the process 400 may be repeated for each voxel of the initial ray for which shadowing is desired to be performed.

As described herein, the amount that the light source is occluded from the voxel of the initial ray may be determined by performing the process 454 illustrated in FIG. 4B. FIG. 4B illustrates an example process for determining an amount a light source is occluded from a voxel, in accordance with some embodiments of the technology described herein.

The amount by which a light source is occluded from a voxel of the initial ray may depend on the transparency of voxels of the supplemental ray. In order to determine how transparent or opaque the voxels of the supplemental ray are, a value for totalAlpha for each voxel of the supplemental ray may be determined.

Thus, process 454 may begin at act 404A where values for luminosity and gradient may be obtained at locations along the supplemental ray. The locations along the supplemental ray may be evenly spaced according to any suitable spacing and may be referred to herein as voxels of the supplemental ray. As described herein, the spacing may be adjusted depending on desired image resolution and/or available frame rate. Similar to act 306A of process 300, the sampled values for luminosity and gradient for each voxel of the supplemental ray may be obtained based on the image data.

At act 404B, alpha values for luminosity and gradient may be obtained using the sampled values obtained at act 404A. For example, as in act 306B, the alpha values for each voxel of the supplemental ray may be obtained using one or more look-up tables based on the sampled values obtained at act 306A. The look-up tables may be two separate look-up tables or a single look-up table. As described herein, in some embodiments, there is a separate look-up table for each of luminosity and gradient.

At act 404C, a value for totalAlpha may be obtained based on luminosity and gradient alpha values obtained at act 404B. The value for totalAlpha may be obtained for each voxel of the supplemental ray and may be obtained according to the equation previously described herein, repeated below:

totalAlpha=lumColor.a+gradColor.a−(lumColor.a*gradColor.a)

where lumColor.a and gradColor.a are the alpha values for luminosity and gradient, respectively, obtained via the one or more look-up tables at act 404B. As described herein, totalAlpha may be representative of the transparency of a voxel. For example, a totalAlpha value closer to zero may indicate that the voxel is relatively more translucent, while a totalAlpha value closer to 1 may indicate that the voxel is relatively more opaque. A totalAlpha value equal to zero may indicate that the voxel is completely transparent (e.g., allowing light to pass through the voxel completely), while a totalAlpha value equal to 1 may indicate that the voxel is completely opaque (e.g., blocking all light from passing through the voxel).

At act 404D, the totalAlpha values for each voxel along the supplemental y may be summed. For example, starting from the light source, the totalAlpha values for the respective voxels along the supplemental ray may be summed until either all of the values are summed or the summed totalAlpha value reaches a threshold (e.g., 1, in some embodiments, less than 1 in some embodiments). For example, when the summed totalAlpha value reaches the threshold, this may indicate that the light source is totally occluded from the voxel of the initial ray due to the combined opacity of voxels along the supplemental ray and further summation of additional voxels is not required. The summed totalAlpha value may represent the amount by which the light source is occluded from the voxel of the initial ray.

Thus, act 406 of process 400 may comprise darkening the combinedcolor.rgb value of the voxel of the initial ray obtained at act 306E of process 300 based on the summed totalAlpha value obtained at act 404D. For example, a relatively low summed totalAlpha value may darken the combinedcolor.rgb value by decreasing the respect R, G, and B values by a relatively small amount while a relatively high summed totalAlpha value may decrease the combinedcolor.rgb value by a relatively large amount. In some embodiments, where the summed totalAlpha is 1, the combinedcolor.rgb value is reduced to zero, as the light source is substantially completely occluded from the voxel of the initial ray by the voxels of the supplemental ray.

In some embodiments, the combinedcolor.rgb value obtained at act 306E may be darkened according to the following formula:

diffuseLevel=(shadowLevel*cShadowDiffuse)+cShadowAmbient

combinedColor.rgb=combinedColor.rgb*diffuseLevel

where shadowLevel is the transparency of the shadow with a value of 1.0 being transparent and is equal to 1−totalAlpha, and a value of 0.0 being opaque, cShadowAmbient is a configurable ambient lighting level for shadows with a value of 0.0 being black and 1.0 being the original voxel color, and cShadowDiffuse equal to 1.0−cShadowAmbient.

FIG. 5A illustrates an example medical image 502 obtained using ray tracing, in accordance with some embodiments of the technology described herein. The medical image 502 may be obtained according to the methods described herein, for example, as described with respect to FIGS. 2-4.

In particular, medical image 502 illustrates a cross-sectional view of a brain. As shown in FIG. 5A, the techniques described herein for generating a 3D medical image using ray tracing can render a medical image such that tissues like the surface undulations of the ventricular walls of the brain shown in image 502 appear as they would be in the corresponding anatomical dissection. As described herein, the ability to generate photo-realistic medical images at the point of care may improve physician's ability to treat and/or diagnose patients.

In some embodiments, the medical image 502 is viewed via a graphical user interface. FIG. 5B illustrates an example graphical user interface 500 for viewing and interacting with the example medical image of FIG. 5A, in accordance with some embodiments of the technology described herein. As described herein, for example, with reference to system 100 shown in FIG. 1, in some embodiments the GUI 500 is on a display of a mobile computing device. In some embodiments, the GUI 500 comprises a viewer with an adjustable window.

As shown in FIG. 5B, the GUI 500 may display a medical image, such as image 502. In particular, the GUI 500 may display generated medical images having tissue coloration which makes the two- or 3D color image highly realistic. In some embodiments, a 3D medical image is generated according to the techniques described herein and the processor may output the medical image for display on the GUI 500 in two dimensions. In some embodiments, the 3D medical image may be displayed and viewed via the GUI 500 with use of appropriate equipment for viewing 3D images. In some embodiments, the medical image may be used with extended reality interfaces such as augmented reality and/or virtual reality interfaces. The GUI may be run on a single viewer engine which can handle all different viewing modes, including both 3D and two dimensional image viewing.

The GUI 500 may receive input from a user indicative of a change to be made to a medical image. For example, as shown in FIG. 5B, the GUI 500 comprises a number of options 504-524 for interacting with the medical image. The GUI 500 may comprise controls which are familiar to users of medical image viewers. As such, the GUI 500 may be easily accessible even to untrained users.

Box 504 comprises an option for a user to generate and/or display a new image. In some embodiments, box 504 comprises generating a new medical image according to the ray tracing techniques described herein. In such embodiments, generating the new medical image may be performed in response to receiving input from the user. In some embodiments, generation of a new image may be performed in response to receiving at least a portion of the image data. Thus, image generation using ray tracing may, in some embodiments, be performed in real time.

Box 506 comprises an option to add an additional medical image for display with the GUI 500. Adding an image may comprise generating and/or viewing a second medical image in addition to the first medical image 502. The second medical image may be generated from medical image data of a same or different type as the medical image data used to generate the first medical image. In some embodiments, the first and second medical images may be viewed side by side. In some embodiments, the second medical image may be overlaid on the first medical image. The inventors have recognized that the ability to cross-reference between different types of highly realistic medical images may improve a physician's ability to treat and/or diagnose a patient.

Boxes 508-518 comprise additional options for interacting with the medical image 502. For example, at box 508 a user may translate an object depicted by the medical image. At box 510, a user may rotate the object depicted by the medical image. At box 512, a user may cross-section an object depicted by the medical image by cutting through a plane of the medical image. For example, image 502 illustrates a cross-section of a brain. At box 514, a user may indicate that they wish to view the medical image in 3-D, as described herein. At box 516, a user may zoom in and/or out of portions of the medical image.

According to the techniques developed by the inventors and described herein, the processor may generate an updated 3D medical image in response to receiving the user input indicative of a change to be made to the medical image. For example, when a user rotates the object depicted by the medical image, the processor may repeat the ray tracing process described herein in order to generate an updated image having the object at the rotated position. Thus, the realistic rendering of the object is maintained over time even while the object is being manipulated by a user via the GUI.

At box 518, a user may initiate an interactive viewing mode referred to herein as “rocker” mode. In rocker mode, the user may select a point on the object depicted by the medical image using, for example, a cursor. When rendering a slice of the volume based on a 2D cutting plane that faces the user, the user may drag their mouse or finger starting from the selected point on the 2D plane. This point may become fixed in the view, and the orientation of the cutting plane in volume space may change as the user drags their mouse or finger around the point. Throughout, the volume is reoriented so that the cutting plane continues to face the user. This allows the user to see volume features around the point from where the dragging commenced. The viewer may revert to an original view when the user stops dragging. During rotation and cross-sectioning of the object in rocker mode, the processor may regenerate the medical image at each motion. For example, in response to rotation and/or cross-section of the image during rocker mode, the processor may repeat the ray tracing process described herein in order to generate an updated image.

Boxes 520-524 illustrates additional options available to a user via the GUI. At box 520, the user may annotate the medical image, for example, by inputting notes associated with the image and/or marking up the image. At box 522, the user may save the image and/or associated annotations to a memory (e.g., a memory of the mobile computing device, in some embodiments). At box 524, the user may transmit the image and/or associated annotations to a second device.

As described herein, the GUI may be written completely in JAVASCRIPT and/or GLSL and may be deployable across most major web browsers and operating systems, rapidly over consumer-grade network systems and cellular data networks. In particular, software for performing the techniques described herein and/or rendering the GUI may be executed in a web browser. In some embodiments, software for performing the techniques described herein and/or rendering the GUI may be transmitted over a standard cellular data network.

As described herein, the GUI is extensible with various mouse and/or touchscreen gestures and control widgets on the interface. The control of the viewer is highly accessible to manipulation via application programming interfaces (APIs) of various GUIs. In some embodiments, the GUI can be modified to include other features not specifically described herein, such as on-the-fly smoothing and enhancement of volumes, using algorithms such as anisotropic diffusion filtering or cubic B-spline convolution filtering, and programmatic animation of the volume using the viewer's API, as some illustrative examples.

FIGS. 5C-5E illustrates an example of graphical user interface illustrating medical images in different views, in accordance with some aspects of the technology described herein. As shown in FIG. 5D, the rotation of objects displayed in panes 556A-C of FIG. 5D is different from the rotation of the objects displayed in panes 552A-C of the FIG. 5C. In some embodiments, generating the rotated views in panes 556A-C may include generating updated 3D images to display in panes 556A-C. As shown in FIG. 5E, the objects displayed in panes 550A-C of FIG. 5E have been zoomed in relative to the objects displayed in panes 556A-C of FIG. 5D.

As described herein, a processor may receive image data from at least one medical imaging device. In some embodiments, one or more of the at least one medical imaging devices comprises an MRI device for performing magnetic resonance imaging. The processor may receive MRI data obtained by the MRI device and generate a 3D medical resonance image based on the MRI data. In some embodiments, as described herein, the processor is a processor of a mobile computing device and is in communication with the MRI device. In some embodiments, the processor is integrated with the MRI device, such that the processor is configured to control aspects of the MR imaging by the MRI device in addition to generating the 3D medical image based on image data obtained through the MR imaging. Further aspects of an example MRI device for use in combination with the techniques described herein will now be described.

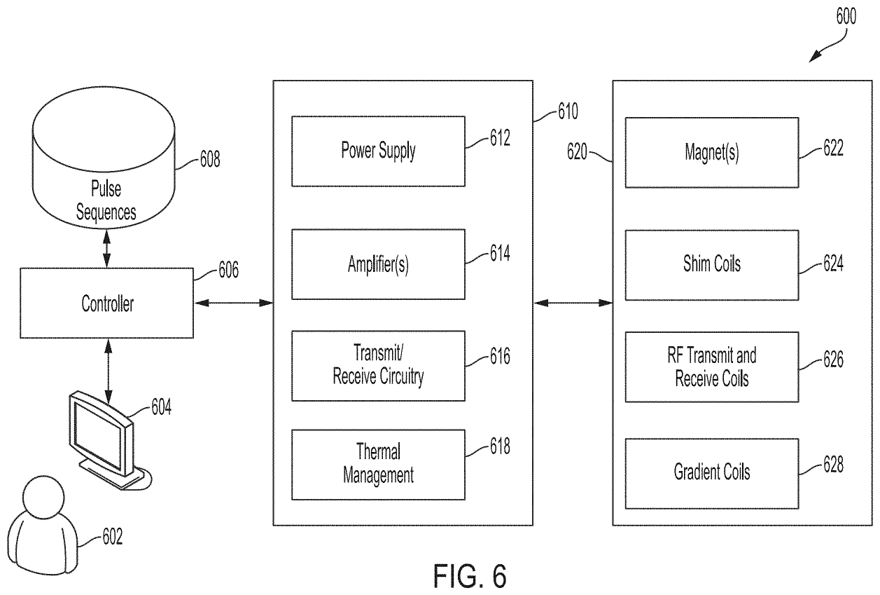

For example, FIG. 6 illustrates exemplary components of an MRI device in accordance with some embodiments. In the illustrative example of FIG. 6, MRI device 600 comprises computing device 604, controller 606, pulse sequences repository 608, power management system 610, and magnetics components 620. It should be appreciated that system 600 is illustrative and that an MRI device may have one or more other components of any suitable type in addition to or instead of the components illustrated in FIG. 6. However, an MRI device will generally include these high-level components, though the implementation of these components for a particular MRI device may differ. Examples of MRI devices that may be used in accordance with some embodiments of the technology described herein are described in U.S. Pat. No.

10,627,464 filed Jun. 30, 2017 and titled “Low-Field Magnetic Resonance Imaging Methods and Apparatus,” which is incorporated by reference herein in its entirety.

As illustrated in FIG. 6, magnetics components 620 comprise B₀ magnets 622, shim coils 624, radio frequency (RF) transmit and receive coils 626, and gradient coils 628. B₀ magnets 622 may be used to generate the main magnetic field B₀. B₀ magnets 622 may be any suitable type or combination of magnetics components that can generate a desired main magnetic B₀ field. In some embodiments, B₀ magnets 622 may be a permanent magnet, an electromagnet, a superconducting magnet, or a hybrid magnet comprising one or more permanent magnets and one or more electromagnets and/or one or more superconducting magnets. In some embodiments, B₀ magnets 622 may be configured to generate a B₀ magnetic field having a field strength that is less than or equal to 0.2 T or within a range from 50 mT to 0.1 T.

For example, in some embodiments, B₀ magnets 622 may include a first and second B₀ magnet, each of the first and second B₀ magnet including permanent magnet blocks arranged in concentric rings about a common center. The first and second B₀ magnet may be arranged in a bi-planar configuration such that the imaging region is located between the first and second B₀ magnets. In some embodiments, the first and second B₀ magnets may each be coupled to and supported by a ferromagnetic yoke configured to capture and direct magnetic flux from the first and second B₀ magnets.

Gradient coils 628 may be arranged to provide gradient fields and, for example, may be arranged to generate gradients in the B₀ field in three substantially orthogonal directions (X, Y, Z). Gradient coils 628 may be configured to encode emitted MR signals by systematically varying the B₀ field (the B₀ field generated by B₀ magnets 622 and/or shim coils 624) to encode the spatial location of received MR signals as a function of frequency or phase. For example, gradient coils 628 may be configured to vary frequency or phase as a linear function of spatial location along a particular direction, although more complex spatial encoding profiles may also be provided by using nonlinear gradient coils. In some embodiments, gradient coils 628 may be implemented using laminate panels (e.g., printed circuit boards).

MRI is performed by exciting and detecting emitted MR signals using transmit and receive coils, respectively (often referred to as radio frequency (RF) coils). Transmit/receive coils may include separate coils for transmitting and receiving, multiple coils for transmitting and/or receiving, or the same coils for transmitting and receiving. Thus, a transmit/receive component may include one or more coils for transmitting, one or more coils for receiving and/or one or more coils for transmitting and receiving. Transmit/receive coils are also often referred to as Tx/Rx or Tx/Rx coils to generically refer to the various configurations for the transmit and receive magnetics component of an MRI device. These terms are used interchangeably herein. In FIG. 6, RF transmit and receive circuitry 616 comprises one or more transmit coils that may be used to generate RF pulses to induce an oscillating magnetic field B₁. The transmit coil(s) may be configured to generate any suitable types of RF pulses.

Power management system 610 includes electronics to provide operating power to one or more components of the low-field MRI device 600. For example, power management system 610 may include one or more power supplies, energy storage devices, gradient power components, transmit coil components, and/or any other suitable power electronics needed to provide suitable operating power to energize and operate components of MRI device 600. As illustrated in FIG. 6, power management system 610 comprises power supply system 612, power component(s) 614, transmit/receive switch 616, and thermal management components 618 (e.g., cryogenic cooling equipment for superconducting magnets, water cooling equipment for electromagnets).

Power supply system 612 includes electronics to provide operating power to magnetic components 620 of the MRI device 600. The electronics of power supply system 612 may provide, for example, operating power to one or more gradient coils (e.g., gradient coils 628) to generate one or more gradient magnetic fields to provide spatial encoding of the MR signals. Additionally, the electronics of power supply system 612 may provide operating power to one or more RF coils (e.g., RF transmit and receive coils 626) to generate and/or receive one or more RF signals from the subject. For example, power supply system 612 may include a power supply configured to provide power from mains electricity to the MRI device and/or an energy storage device. The power supply may, in some embodiments, be an AC-to-DC power supply configured to convert AC power from mains electricity into DC power for use by the MRI device. The energy storage device may, in some embodiments, be any one of a battery, a capacitor, an ultracapacitor, a flywheel, or any other suitable energy storage apparatus that may bidirectionally receive (e.g., store) power from mains electricity and supply power to the MRI device. Additionally, power supply system 612 may include additional power electronics encompassing components including, but not limited to, power converters, switches, buses, drivers, and any other suitable electronics for supplying the MRI device with power.

Amplifiers(s) 614 may include one or more RF receive (Rx) pre-amplifiers that amplify MR signals detected by one or more RF receive coils (e.g., coils 626), one or more RF transmit (Tx) power components configured to provide power to one or more RF transmit coils (e.g., coils 626), one or more gradient power components configured to provide power to one or more gradient coils (e.g., gradient coils 628), and one or more shim power components configured to provide power to one or more shim coils (e.g., shim coils 624). Transmit/receive switch 616 may be used to select whether RF transmit coils or RF receive coils are being operated.

As illustrated in FIG. 6, MRI device 600 includes controller 106 (also referred to as a console) having control electronics to send instructions to and receive information from power management system 610. Controller 606 may be configured to implement one or more pulse sequences, which are used to determine the instructions sent to power management system 610 to operate the magnetic components 620 in a desired sequence (e.g., parameters for operating the RF transmit and receive coils 626, parameters for operating gradient coils 628, etc.). As illustrated in FIG. 6, controller 606 also interacts with computing device 604 programmed to process received MR data. For example, computing device 604 may process received MR data to generate one or more MR images using any suitable image reconstruction process(es). Controller 106 may provide information about one or more pulse sequences to computing device 604 for the processing of data by the computing device. For example, controller 606 may provide information about one or more pulse sequences to computing device 604 and the computing device may perform an image reconstruction process based, at least in part, on the provided information.

Computing device 604 may be any electronic device configured to process acquired MR data and generate one or more images of a subject being imaged. In some embodiments, computing device 604 may be located in a same room as the MRI device 600 and/or coupled to the MRI device 600. In some embodiments, computing device 604 may be a fixed electronic device such as a desktop computer, a server, a rack-mounted computer, or any other suitable fixed electronic device that may be configured to process MR data and generate one or more images of the subject being imaged. Alternatively, computing device 604 may be a portable device such as a smart phone, a personal digital assistant, a laptop computer, a tablet computer, or any other portable device that may be configured to process MR data and generate one or images of the subject being imaged. In some embodiments, computing device 604 may comprise multiple computing devices of any suitable type, as aspects of the disclosure provided herein are not limited in this respect.

The exemplary low-field MRI devices described above and in U.S. Pat. No. 10,627,464 can be used to obtain image data which may be used to generate 3D medical images according to the ray tracing techniques described herein. The inventors have recognized that the techniques described herein for generation of 3D medical images may be used in combination with the portable low-field MRI devices to improve the ability of a physician to treat and/or diagnose patients. For example, a patient may undergo an MR scan at the point-of-care to obtain MR image data. A device (such as a mobile computing device) may receive the MRI image data and use the data to generate a 3D medical image in real time, at the patient's bedside, for example. The 3D medical image may depict a photo-realistic rendering of the patient anatomy that was imaged. The physician may manipulate the generated image, via a GUI, as described herein. For example, the physician may translate, rotate, cross-section, and/or zoom in or out of the object. The physician may cross-reference the generated image with one or more other images. Although example MRI devices are described herein and in U.S. Pat. No. 10,627,464, any suitable type of MRI device may be used in combination with the techniques described herein, including, for example high-field MRI devices.

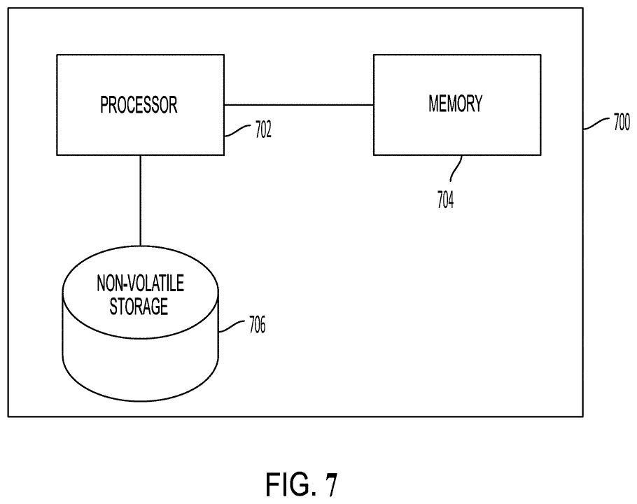

FIG. 7 shows a block diagram of an example computer system 700 that may be used to implement embodiments of the technology described herein. The computing device 700 may include one or more computer hardware processors 702 and non-transitory computer-readable storage media (e.g., memory 704 and one or more non-volatile storage devices 706). The processor(s) 702 may control writing data to and reading data from (1) the memory 704; and (2) the non-volatile storage device(s) 706. To perform any of the functionality described herein, the processor(s) 702 may execute one or more processor-executable instructions stored in one or more non-transitory computer-readable storage media (e.g., the memory 704), which may serve as non-transitory computer-readable storage media storing processor-executable instructions for execution by the processor(s) 702.

Having thus described several aspects and embodiments of the technology set forth in the disclosure, it is to be appreciated that various alterations, modifications, and improvements will readily occur to those skilled in the art. For example, although aspects of the technology are described herein with reference to generating 3D medical images, it should be appreciated that the techniques may be extended for use in any suitable application. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor, but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated. that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “substantially”, “approximately”, and “about” may be used to mean within ±20% of a target value in some embodiments, within ±10% of a target value in some embodiments, within ±5% of a target value in some embodiments, within ±2% of a target value in some embodiments. The terms “approximately” and “about” may include the target value.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. A system for generating a three-dimensional (3D) medical image, the system comprising:
a mobile computing device comprising at least one computer hardware processor; and
at least one non-transitory computer readable storage medium having encoded thereon executable instructions that, when executed by the at least one computer hardware processor, cause the mobile computing device to perform:
receiving, via at least one communication network, image data obtained by at least one medical imaging device;
generating, using ray tracing, the 3D medical image based on the image data obtained by the at least one medical imaging device; and
outputting the 3D medical image.

2. The system of claim 1, wherein the at least one medical imaging device comprises a magnetic resonance imaging (MRI) device, and wherein receiving the image data comprises receiving MRI data obtained by the MRI device while imaging a subject.

3. The system of claim 1, wherein outputting the 3D medical image comprises displaying the 3D medical image on the mobile computing device, transmitting the 3D medical image to a second device external to the mobile computing device for viewing by a user of the second device, and/or saving the 3D medical image to a memory of the mobile computing device.

4. The system of claim 1, wherein the executable instructions comprise JAVASCRIPT and/or GL SHADING LANGUAGE instructions for generating the 3D medical image using ray tracing.

5. The system of claim 4, wherein the generating is performed by executing the JAVASCRIPT and/or GL SHADING LANGUAGE instructions on a web browser executing on the mobile computing device.

6. The system of claim 1, wherein the generating is performed in response to receiving at least a portion of the image data.

7. The system of claim 1, wherein performing the ray tracing comprises:
generating at least one ray;
determining values for at least one characteristic at locations along the at least one ray ray; and
generating a pixel value based on the determined values for the at least one characteristic at the locations along the at least one ray.

8. The system of claim 1, wherein the executable instructions further cause the mobile computing device to generate a graphical user interface (GUI) for viewing the 3D medical image, wherein the GUI is configured to allow a user to provide an input indicative of a change to be made to the 3D medical image.

9. The system of claim 8, wherein the executable instructions further cause the mobile computing device to generate, using ray tracing, an updated 3D medical image in response to receiving the input provided by the user.

10. The system of claim 1, wherein the mobile computing device is battery-powered.

11. A method for generating a three-dimensional (3D) medical image comprising:
receiving, with at least one computer hardware processor of a mobile computing device via at least one communication network, image data obtained by at least one medical imaging device;
generating, using the at least one computer hardware processor to perform ray tracing, the 3D medical image based on the image data obtained by the at least one medical imaging device; and
outputting the 3D medical image.

12. The method of claim 11, wherein the at least one medical imaging device comprises a magnetic resonance imaging (MRI) device, and wherein receiving the image data comprises receiving MRI data obtained by the MRI device.

13. The method of claim 11, wherein outputting the 3D medical image comprises displaying the 3D medical image on the mobile computing device, transmitting the 3D medical image to a second device external to the mobile computing device for viewing by a user of the second device, and/or saving the 3D medical image to a memory of the mobile computing device.

14. The method of claim 11, wherein the generating is performed by executing, with the at least one computer hardware processor on a web browser executing on the mobile computing device, JAVASCRIPT and/or GL SHADING LANGUAGE instructions for generating the 3D medical image using ray tracing.

15. The method of claim 11, further comprising generating a graphical user interface (GUI) for viewing the 3D medical image and being configured to allow a user to provide an input indicative of a change to be made to the 3D medical image, wherein the method further comprises generating, using ray tracing, an updated 3D medical image in response to receiving the input provided by the user.

16. At least one non-transitory computer readable storage medium having encoded thereon executable instructions that, when executed by at least one computer hardware processor of a mobile computing device, cause the mobile computing device to perform:
receiving, via at least one communication network, image data obtained by at least one medical imaging device;
generating, using ray tracing, a three-dimensional (3D) medical image based on the image data obtained by the at least one medical imaging device; and
outputting the 3D medical image.

17. The at least one non-transitory computer-readable storage medium of claim 16, wherein the at least one medical imaging device comprises a magnetic resonance imaging (MRI) device, and wherein receiving the image data comprises receiving MRI data obtained by the MRI device.

18. The at least one non-transitory computer-readable storage medium of claim 16, wherein outputting the 3D medical image comprises displaying the 3D medical image on the mobile computing device, transmitting the 3D medical image to a second device external to the mobile computing device for viewing by a user of the second device, and/or saving the 3D medical image to a memory of the mobile computing device.

19. The at least one non-transitory computer-readable storage medium of claim 16, wherein the executable instructions comprise JAVASCRIPT and/or GL SHADING LANGUAGE instructions for generating the 3D medical image using ray tracing, and the generating is performed by executing the JAVASCRIPT and/or GL SHADING LANGUAGE instructions on a web browser executing on the mobile computing device.

20. The at least one non-transitory computer-readable storage medium of claim 16, wherein the executable instructions further cause the mobile computing device to generate a graphical user interface (GUI) for viewing the 3D medical image and being configured to allow a user to provide an input indicative of a change to be made to the 3D medical image, wherein the executable instructions further cause the mobile computing device to generate, using ray tracing, an updated 3D medical image in response to receiving the input provided by the user.
